SEQUENCE LISTING (2 sequences)

SEQ ID NO: 1 - protein, 4 aa, Artificial Sequence
  Peptide
RRGW

SEQ ID NO: 2 - protein, 8 aa, Artificial Sequence
  Peptide
CCGGRRGW

(19) United States
(12) Patent Application Publication
(10) Pub. No.: US 2023/0349910 A1
(43) Pub. Date: Nov. 2, 2023

(54) FLUORESCENT REPORTER AND USE THEREOF FOR THE DETECTION OF TARGET MOLECULES

(71) Applicants: DYAMEO, Limoges (FR); KAMAX INNOVATIVE SYSTEM, Limoges (FR); UNIVERSITÉ DE LIMOGES, Limoges (FR)

(72) Inventors: Alexis SAINTAMAND, Limoges (FR); Virgile BARRET-VIVIN, Limoges (FR); Cédric ENGUEHARD, Limoges (FR); Jérôme DESROCHES, Couzeix (FR); Akil HUSSEIN, Masseret (FR); Nicolas VEDRENNE, Rezé (FR)

(73) Assignees: DYAMEO, Limoges (FR); KAMAX INNOVATIVE SYSTEM, Limoges (FR); UNIVERSITÉ DE LIMOGES, Limoges (FR)

(21) Appl. No.: 18/044,833

(22) PCT Filed: Sep. 10, 2021
(86) PCT No.: PCT/FR2021/051554

§ 371 Date: Mar. 10, 2023

(30) Foreign Application Priority Data
Sep. 11, 2020 (FR) 2009245

(51) Int. Cl. G01N 33/58; G01N 33/68; G01N 21/64

(52) U.S. Cl. CPC G01N 33/582; G01N 33/6803; G01N 21/6428

(57) ABSTRACT

A device for detecting a target molecule and/or measuring the concentration of a target molecule, which includes: a substrate at the surface of which is covalently attached a grafting molecule; at least one fluorescent probe including at least one receptor bonded to a polypeptide via a covalent bond; two fluorochromes Fₐ and F_{b}, in which the fluorochrome Fₐ is bonded to the receptor and fluorochrome F_{b} is bonded to the polypeptide; and the fluorochromes Fₐ and F_{b} form a FRET donor/acceptor pair, in which the polypeptide is bonded to the grafting molecule via a covalent bond. Also, a fluorescent probe and a method for detecting a target molecule and/or measuring the concentration of a target molecule.

### FIELD OF THE INVENTION

The present invention relates to a fluorescent reporter, or fluorescent probe, for detecting and/or measuring the concentration of a target molecule in a sample.

### PRIOR ART

The detection of target molecules in a sample has become essential to screen for contaminants in agri-food products, in wastewater or for medical research such as for example for the diagnosis of many pathological conditions, including cancers, infectious diseases, autoimmune diseases and allergies. The detection of target molecules using FRET technology, based on non-radiative energy transfer between two fluorochromes, conventionally requires a FRET donor/acceptor pair of which each individual element carries a recognition molecule such as an antibody. This fluorescent reporter, i.e. fluorescent probe, in two parts has certain drawbacks: the two parts must recognise the target molecule to generate the FRET effect and therefore the detection of the target molecule, the detection is long, and the sensitivity thereof is fundamentally limited by the concentration of the target molecule and the affinity of the antibodies. The development of a fluorescent probe based on intramolecular FRET would make it possible to avoid some of these constraints.

Such fluorescent probes are known from the prior art. Particularly the documents Grant et al. (Grant et al., “Effects of immobilization on a FRET immunosensor for the detection of myocardial infarction”, Anal Bioanal Chem (2005), 381: 1012-1018) and Ko et al. (Ko et al., “A novel FRET-based optical fiber biosensor for rapid detection of Salmonella typhimurium”, Biosensors and Bioelectronics (2006), 21: 1283-1290) describe a fluorescent probe comprising an antibody and a protein A bonded to two fluorochromes forming a FRET donor/acceptor pair. The antibody and protein A are not bonded by covalent bonding, resulting in a risk of separation of the fluorescent probe during the detection of a target molecule and therefore preventing the use thereof for in vivo detection. A second consequence of the weak antibody-protein A bond is the high detection threshold.

The document FR 3 040 789 moreover describes a fluorescent probe comprising two antibodies each labelled by a member fluorochrome of a FRET donor/acceptor pair. These two antibodies are not bonded to each other and do not make it possible to obtain an intramolecular FRET effect.

In parallel, the in vivo application of fluorescent probes remains very limited. In this context, the methods used are generally based on the injection of non-specific tracers or antibodies coupled with a fluorochrome. Detection is then performed by measuring the intensity of the signal present on the surface of the tissues. These approaches, although sometimes used, have numerous drawbacks. In particular, the signal obtained is strongly affected by the stability of the probe in vivo, the bioavailability thereof or the specificity thereof. Furthermore, the safety of this injectable product must be demonstrated systematically. The development of a fluorescent probe composed of a single molecule reacting to the presence of a target molecule with a change of optical properties would enable the detection of markers of interest by mere contact, without the constraints associated with injection.

During in vivo use, for example during surgery or an exploration of a part of the human body by endoscopy, it is crucial for the surgeon to be able to identify tumour cells present in tissues with certainty and rapidity. Thus, fluorescence-assisted surgery is currently a growing field, but is limited by the inherent defects of marker injection.

Therefore, there is a real need in respect of fluorescent probes capable of being used during surgery or with endoscopy, at the end of the optical fibre for example, without any risk of biological contamination, and making it possible to confirm the diagnosis.

During a use in vitro, for example during the rapid analysis of solutions for medical diagnostic purposes or for the purposes of target molecules in an environmental sample, it is necessary to avoid biological contamination of the sample under study by the fluorescent probe as is the case with the fluorescent probes developed to date. The present invention proposes a solution to this problem by providing a fluorescent probe wherein the different elements are bonded strongly to each other, particularly the receptor part so that it does not separate from the other elements forming the fluorescent probe in aid of the target molecule to be detected, inducing a biological contamination of the sample. Furthermore, the fluorescent probe according to the invention has the advantage of not requiring any additional step of type handling, washing, secondary labelling or other. Merely placing it in contact with a sample to be analysed is sufficient, which considerably reduces the number of handling operations of the sample, as well as the time for obtaining the results.

### SUMMARY

The present invention relates to a device for detecting a target molecule and/or measuring the concentration of a target molecule comprising:
a substrate at the surface of which a grafting molecule is covalently attached;
at least one fluorescent probe comprising:
   at least one receptor bonded to a polypeptide via a covalent bond;
   two fluorochromes Fₐ and F_{b};
      wherein the fluorochrome Fₐ is bonded to the receptor and the fluorochrome F_{b} is bonded to the polypeptide; and
      the fluorochromes Fₐ and F_{b} form a FRET donor/acceptor pair;
      wherein the polypeptide is bonded to the grafting molecule via a covalent bond.

According to an embodiment, the receptor is chosen from antibodies, antibody fragment, aptamer, proteins, peptides, or a derivative thereof. According to an embodiment, the polypeptide is a binding protein chosen from protein G, protein L, protein A, protein Z, protein M, immunoglobulin, a complete or partial immunoglobulin, or a derivative thereof. According to an embodiment, the polypeptide comprises between 2 and 100 amino acids, preferably between 4 and 50 amino acids. According to an embodiment, the fluorochromes Fₐ and/or F_{b} are chosen from fluorescent molecules or fluorescent proteins. According to an embodiment, the substrate is chosen from a cell culture plate, a well plate, a film, a strip, an agarose gel, a cellulose gel, nanoparticles or microparticles, preferably spherical, preferably silica or polymer, a microscope slide, a glass strip, the periphery of an optical fibre or a substrate configured to be attached to head of an optical fibre. According to an embodiment, the substrate is a polymer film. According to an embodiment, the polymer film is chosen from polyethylene terephthalate, fluorinated polyethylene-co-propylene, polymethylmethacrylate, polytetrafluoroethylene, polymethylpentene, polyvinyl chloride, styrene methyl methacrylate, polyethylene naphthalate, derivatives thereof or a mixture thereof. According to an embodiment, the grafting molecule comprises at least two reactive groups chosen from maleimide, N-Hydroxysuccinimide (NHS) ester, sulfo N-hydroxysuccinimide ester, sulfo-NHS, azide, alkyne, epoxide, carboxylic acid, aldehyde, aziridine, alkene, or a derivative thereof. According to an embodiment, the device further comprises an optical fibre and an exploration head, wherein said exploration comprises a body and an emission face of which at least a part is transparent forming a port, the substrate being said port.

The present invention also relates to a fluorescent probe comprising:
at least one receptor bonded to a polypeptide via a covalent bond;
two fluorochromes Fₐ and F_{b};
   wherein the fluorochrome Fₐ is bonded to the receptor and the fluorochrome F_{b} is bonded to the polypeptide; and
   the fluorochromes Fₐ and F_{b} form a FRET donor/acceptor pair.

According to an embodiment, the receptor is chosen from antibodies, antibody fragment, aptamers, proteins, peptides, or a derivative thereof. According to an embodiment, the polypeptide is a binding protein chosen from protein G, protein L, protein A, protein Z, protein M, immunoglobulin, a complete or partial immunoglobulin, or a derivative thereof. According to an embodiment, the polypeptide comprises between 2 and 100 amino acids, preferably between 4 and 50 amino acids.

The present invention also relates to a method for detecting a target molecule and/or measuring the concentration of a target molecule comprising the following steps:
Placing a sample and at least one fluorescent probe in contact, said fluorescent probe comprising:
   at least one receptor bonded to a polypeptide via a covalent bond;
   two fluorochromes Fₐ and F_{b};
      wherein the fluorochrome Fₐ is bonded to the receptor and the fluorochrome F_{b} is bonded to the polypeptide; and
      the fluorochromes Fₐ and F_{b} form a FRET donor/acceptor pair; and
      the receptor has an affinity for said target molecule;
Exciting the fluorescent probe at a given wavelength such that the donor fluorochrome is excited;
Measuring the ratio between the intensity of the fluorescence emitted by the donor fluorochrome and the intensity of the fluorescence emitted by the acceptor fluorochrome; and
Determining the presence or the absence of said target molecule in the sample and/or calculating the concentration of said target molecule in the sample.

### DEFINITIONS

In the present invention, the terms below are defined as follows:

“Antibodies” (also known as immunoglobulins, abbreviated to Ig) relate to gamma globulin proteins found in the blood or other bodily fluids of vertebrates and are used by the immune system to identify and neutralise foreign bodies, such as bacteria and viruses. Antibodies consist of two pairs of polypeptide chains, referred to as heavy chains and light chains arranged in a Y shape. The two ends of the Y are the regions which bond to antigens and deactivate them. The term “antibody” (Ab) as used here comprises monoclonal antibodies, polyclonal antibodies, multispecific antibodies (for example bispecific antibodies). The term “immunoglobulin” (Ig) is used interchangeably with “antibody”.

“Antigen” refers to a molecule which induces an immune response. This immune response can involve either antibody production, or the activation of specific immunologically competent cells, or both. A person skilled in the art will understand that any macromolecule, including practically any proteins or any peptides, can serve as antigen.

“Aptamer” relates to a synthetic oligonucleotide, generally an RNA which is capable of binding a specific ligand.

“Active configuration” (annotated “ON”) refers to the configuration of the fluorescent probe in the presence of energy transfer (FRET effect) between the fluorochromes Fₐ and F_{b}.

“Inactive configuration” (annotated “OFF”) refers to the configuration of the fluorescent probe in the absence of energy transfer (FRET effect) between the fluorochromes Fₐ and F_{b}.

“Fluorochrome” (or fluorophore) relates to a chemical substance capable of emitting fluorescent light after excitation.

“Antibody fragment” comprises a part of an intact antibody, and particularly includes the variable part responsible for specific antigen recognition. Examples of antibody fragments comprise Fab, Fab′, (Fab′)₂ and Fv, scFv, scFv-Fc fragments; dimeric antibody fragments; linear antibodies (see U.S. Pat. No. 5,641,870; Zapata et al., Protein Eng. 8 (10): 1057-1062 [1995]); single-stranded antibody molecules; and multispecific antibodies formed from antibody fragments. The expression “antibody fragment” (or functional fragment) is a compound having a qualitative biological activity in common with a full-length antibody. Papain digestion of the antibodies produces two identical antigen-binding fragments, referred to as “Fab” fragments, and a residual “Fc” fragment, a term reflecting the ability to crystallise easily. The Fab fragment is composed of an entire L chain with a variable region domain of the H chain (VH) and the first constant domain of a heavy chain (CH1). Each Fab fragment is monovalent with respect to antigen binding, i.e. it has a single antigen-binding site. Treating an antibody with pepsin gives a single large (Fab′)₂ fragment which corresponds approximately to two Fab fragments bonded by a disulphide bridge having a divalent antigen-binding activity and which is always capable of crosslinking the antigen. Fab′ fragments differ from Fab fragments by having some additional residues at the carboxy terminus of the CH1 domain comprising one or more cysteines of the hinge region of the antibody. Fab′-SH is the term here for Fab′ wherein the cysteine residue(s) of the constant domains carry a free thiol group. The F(ab′)₂ antibody fragments were initially produced in the form of pairs of Fab′ fragments which have hinge cysteines therebetween. Further chemical antibody fragment couplings are also known.

“Ligand” relates to a specific target molecule capable of binding reversibly with a receptor. The ligand interacts non-covalently and specifically with said receptor. The bond is formed thanks to the forces between molecules, such as ionic bonds, hydrogen bonds and van der Waals forces. An antibody/antigen pair is an example of receptor/ligand pair. In the present description, the terms “ligand”, “antigen” and “target molecule” are interchangeable.

“Optically transparent” relates to a material which absorbs less than 50%, preferably less than 20%, more preferably less than 10% of light at the wavelength between 350 nm and 1100 nm.

“Polypeptide” relates to a chain of amino acids connected by peptide bonds. This definition includes amino acid chains comprising between 1 and 100 amino acids and amino acid chains comprising more than 100 amino acids, more commonly referred to as proteins.

“Protein” refers to a functional entity formed of one or more peptides. It consists of a polypeptide comprising more than 100 amino acids.

“Protein G” refers to a surface protein expressed by certain streptococcus strains. It binds with a high affinity with Fc fragments of immunoglobulins of different classes of a large number of species. It binds particularly with all human, mouse, rate IgG subtypes, and those of numerous other mammalian species. It preferably binds with Fc fragments, but can also bind with Fab fragment. Due to the affinity therefore for the Fc region of immunoglobulins of numerous mammalian species, protein G is now considered as a universal reagent in biochemistry and immunology.

“Fluorescent reporter” relates to an entity having fluorescence properties for detecting a specific target molecule (or ligand), i.e., relates to a fluorescent probe. Such an entity can for example comprise a receptor-polypeptide pair as described hereinafter. The terms “fluorescent reporter”, “fluorescent biosensor” and “fluorescent probe” are used interchangeably hereinafter.

“Receptor” relates to a biological molecule capable of recognising and/or binding reversibly with a specific target molecule (or ligand). The receptor interacts non-covalently and specifically with said target molecule. The bond is formed thanks to the forces between molecules, such as ionic bonds, hydrogen bonds and van der Waals forces. An antibody/antigen pair is an example of receptor/ligand pair.

### DETAILED DESCRIPTION

### Fluorescent Probe

The present invention relates to a fluorescent probe (also referred to fluorescent reporter) comprising:
at least one receptor bonded to a polypeptide via a covalent bond;
two fluorochromes Fₐ and F_{b}.

The fluorochrome Fₐ is bonded to the receptor and the fluorochrome F_{b} is bonded to the polypeptide. The fluorochromes Fₐ and F_{b} form a FRET donor/acceptor pair.

Thus, the fluorescent probe comprises a part responsible for the specific binding of a target molecule to be detected (receptor), two fluorochromes capable of converting the recognition of the target molecule into a measurable fluorescent signal (Fₐ and F_{b}) and a support system for one of the two fluorochromes also capable of serving as an attachment enabling a control bond on a substrate (polypeptide).

During the recognition of a target molecule by the fluorescent probe, a conformational change of the receptor takes place. This conformational change affects the relative positions of the VH (variable heavy chain) and VL (variable light chain) variable fragments, as well as constant fragments, modifying the distance between the two fluorochromes. This results in variations of the emissions levels of the donor and acceptor fluorochromes by a non-radiative energy transfer, i.e. FRET (Förster Resonance Energy Transfer) effect. The non-radiative energy transfer enables a modification of the optical signature of the fluorescent probe in the event of modification of the distance between the two fluorochromes. This results in a variation in the fluorescence intensity emitted by each of the two fluorochromes, thus converting the conformational change of the receptor induced by the recognition of the target molecule into a measurable fluorescent signal. Thus, it is possible to detect a target molecule in vitro or in vivo thanks to the fluorescent signal emitted by the probe.

To form a FRET donor/acceptor pair, the two fluorochromes Fₐ and F_{b} must have compatible spectral characteristics, particularly an overlap of the emission spectrum of the so-called “donor” fluorochrome with the excitation spectrum of the so-called “acceptor” fluorochrome. When the donor fluorochrome is excited, the fluorescence thereof will then make it possible to excite the acceptor fluorochrome. The efficiency of this energy transfer is essentially dependent on the distance between the two fluorochromes, the extinction coefficient thereof and the quantum yield thereof, as well as the extent of the overlap between the emission and excitation spectrum thereof.

The specific locations of the fluorochromes on the receptor and the polypeptide are optimised to promote changes of the optical signature thereof in fluorescence in the event of recognition and/or binding of the target molecule. Preferably, the fluorochrome F_{b} is grafted on a free amine of the polypeptide.

The polypeptide has a certain affinity for the receptor, for example the receptor is an antibody and the binding protein is a protein G.

The covalent bond between the receptor and the polypeptide is stronger than the receptor-target molecule bond formed on the recognition of said target molecule by the receptor. This makes it possible to ensure that, once the target molecule has been recognised, the receptor will not detach from the polypeptide. Thus the separation of the fluorescent probe into two parts is avoided. Preventing the separation between the receptor and the polypeptide is particularly important when the fluorescent probe is used for the in vivo detection of target molecules, particularly when it is grafted at the distal end of an optical fibre with a view to intracorporeal exploration, as this limits the risk of leaving a part of the fluorescent probe (that with the receptor) in the patient's body when the fibre is removed. Furthermore, without being bound by any theory, the applicant observed that a covalent bond between the receptor and the polypeptide improves the efficiency of the FRET effect, particularly the detection threshold of a target molecule is lower in the case of a fluorescent probe comprising a receptor and polypeptide bonded via a covalent bond, indicating a significant improvement in the sensitivity of the fluorescent probe.

According to an embodiment, the receptor is bonded to the polypeptide via a hetero or monobifunctional binding molecule (“crosslinker”). Preferably, the binding molecule has two or more reactive groups, chosen from: carboxyl-to-amine reactive groups such as, for example, carbodiimide; amine reactive groups such as, for example, NHS ester, imidoester, pentafluorophenyl ester, hydroxymethyl phosphine; sulfhydryl reactive groups such as, for example, maleimide, haloacetyl (bromo- or iodo-), pyridyldisulfide, thiosulfonate, vinylsulfone); aldehyde reactive groups such as, for example, hydrazide, alkoxyamine; photoreactive groups such as, for example, diazirine, aryl azide, hydroxyl (non-aqueous) reactive groups such as, for example, isocyanate).

In a preferred configuration of this embodiment, the binding molecule is chosen from glutaraldehyde, formaldehyde, disuccinimidyl tartrate, tris(hydroxymethyl) phosphine, 1-Ethyl-3-(3-dimethylaminopropyl) carbodiimide, N-Hydroxysuccinimide, bis(sulfosuccinimidyl)suberate, 1,3-Butadiendiepoxide, succinimidyl iodoacetate, succinimidyl (4-iodoacetyl)aminobenzoate, sulfosuccinimidyl (4-iodoacetyl)aminobenzoate, a mixture thereof or a derivative thereof. Preferably, the binding molecule is chosen from glutaraldehyde, succinimidyl iodoacetate, succinimidyl (4-iodoacetyl)aminobenzoate or sulfosuccinimidyl (4-iodoacetyl)aminobenzoate.

According to an embodiment, the covalent bond between the antibody and the binding protein can be obtained by photoactivation of a modified amino acid containing photo-inducible reactive group. In a preferred configuration of this embodiment, the modified amino acid can be a photo-leucine or photo-methionine, the reactive group being a diazirine or an aryl azide.

According to an embodiment, the receptor is chosen from antibody, antibody fragment, aptamer, peptides, or a derivative thereof.

According to an embodiment, the receptor is capable of recognising and/or binding reversibly with a ligand, i.e. a target molecule. The ligand corresponds to any molecule for which the receptor has an affinity and a strong specificity, and capable of binding reversibly with a given receptor.

In a specific configuration of this embodiment, the target molecule (or ligand) is an antigen.

In a specific configuration of this embodiment, the antibody or the antibody fragment is chosen from Fab, Fab′, (Fab′)₂, scFv, or scFv-Fc.

According to an embodiment, the polypeptide comprises a terminal group chosen from thiol, amine, azide, alkyne, epoxide, carboxylic acid, aldehyde, aziridine, alkene, or a derivative thereof.

According to an embodiment, the polypeptide is a binding protein. This enables a fine control of the grafting position of the fluorochrome in the peptide chain. The distance between the polypeptide and the fluorochrome can be modulated via the insertion of a linker.

According to a first specific configuration of this embodiment, the binding protein is preferably an immunoglobulin-binding protein.

According to a specific , the binding protein is chosen from protein A, protein G, protein L, protein M, protein Z, immunoglobulin, a complete or partial immunoglobulin, or a derivative thereof.

According to an embodiment, the polypeptide comprises between 2 and 100 amino acids, preferably between 4 and 50 amino acids, preferably between 5 and 20 amino acids, preferably between 5 and 10 amino acids, even more preferably 8 amino acids. The use of such a polypeptide has numerous advantages:
the fluorescent labelling is better controlled;
   Indeed, it is possible with such a polypeptide to control the number of antibodies bonded to a polypeptide, i.e. bind a single antibody, or a small number of antibodies, to a polypeptide by modulating the number of binding sites capable of accommodating the receptor. This makes it possible to finely control the donor fluorochrome/acceptor fluorochrome ratio.
the sensitivity of the fluorescent probe is enhanced;
   Indeed, it is possible to modulate the distance between the fluorochromes Fₐ and F_{b} by modulating the number of amino acids making up the peptide chain or by modifying the position of the binding site of the receptor on the peptide chain.
the fluorescent probe has superior flexibility;
   Indeed, the steric size is limited in this case.

According to a first specific configuration of this embodiment, the polypeptide is a linear or circular polypeptide. Preferably, the polypeptide comprises the amino acid sequence as described in SEQ ID NO:1 (RRGW). These amino acids form units bonding the Ig.

According to a second specific configuration of this embodiment, the polypeptide comprises 8 amino acids of which the amino acid sequence as described in SEQ ID NO:1 (RRGW). More preferably, the polypeptide comprises the amino acid sequence as described in SEQ ID NO:2 (CCGGRRGW). Even more preferably, the polypeptide comprises the sequence of 8 amino acids as described in SEQ ID NO:2 (CCGGRRGW).

According to an embodiment, the polypeptide can be replaced by an aptamer. In this case, the aptamer has an affinity for the antibody, i.e. the aptamer is capable of binding specifically to the constant part of said antibody to immobilise the latter.

According to a preferred embodiment, the fluorochrome Fₐ is the donor and the fluorochrome F_{b} is the acceptor of the FRET donor/acceptor pair.

According to a further embodiment, the fluorochrome Fₐ is the acceptor and the fluorochrome F_{b} is the donor of the FRET donor/acceptor pair.

According to an embodiment, the fluorochromes Fₐ and/or F_{b} have a fluorescent emission peak between 350 nm and 399 nm (in the UV range), between 400 nm and 499 nm (in the blue range of the visible spectrum), between 500 nm and 599 nm (in the green range of the visible spectrum) or between 600 nm and 719 nm (in the red range of the visible spectrum), between 720 nm and 850 nm (in the near infrared range).

According to an embodiment, the fluorescence emission peaks of the fluorochromes Fₐ and F_{b} have an overlap zone. Advantageously, this overlap enables a non-radiative energy transfer between the two fluorochromes.

According to an embodiment, the fluorochromes Fₐ and/or F_{b} are chosen from fluorescent molecules or fluorescent proteins.

In a specific configuration of this embodiment, a fluorescent molecule is chosen from rhodamine, coumarin, evoblue, oxazine, carbopyronine, naphthalene, biphenyl, anthracene, phenanthrene, pyrene, carbazole, xanthene, cyanin, fluorescein, squaraine, squaraine rotaxane, oxadiazole, acridine, arylmethine, tetrapyrrole, dipyrromethene, or any other fluorescent derivative thereof.

In a specific configuration of this embodiment, a fluorescent protein is chosen from Green Fluorescent Protein (GFP), 22G, aceGFP, amFP486 (“GFP-like fluorescent chromoprotein amFP486”, “Anemonia manjano FP486”), amm2CP, avGFP, AvicFP1, cFP484 (“GFP-like fluorescent chromoprotein cFP484”, “Clavularia cFP484”), dendFP, dfGFP (“Green fluorescent protein”), DrCBD, DsRed, EosFP (“Green to red photoconvertible GFP-like protein EosFP”), eqFP578 (“Red fluorescent protein eqFP578”, “Entacmaea quadricolor FP578”), eqFP611 (“Red fluorescent protein eqFP611”, “Entacmaea quadricolor FP611”), HcRed (“GFP-like non-fluorescent chromoprotein”, “Heteractis crispa Red”), KikG, KO, LanYFPn, Montipora sp20 (“Cytochrome c oxidase subunit 1”), mRed7 (“Rod shape-determining protein MreD”, “mine drainage metagenome 7”), NpR3784g, RpBphP1 (“Rhodopseudomonas palustris BphP1”), RpBphP2 (“Rhodopseudomonas palustris BphP2”), RpBphP6 (“Rhodopseudomonas palustris BphP6”), TeAPCalpha, zFP538 (“GFP-like fluorescent chromoprotein FP538”, “Zoanthus FP538”), AausFP1, vsfGFP-0, LanYFP, bfloGFPa1, RRvT, dLanYFP, dVFP, ccalYFP1, efasGFP, pcDronpa (Green), aeurGFP, Skylan-S (On), mVenus-Q69M, tdTomato, PlamGFP, eechGFP1, mNeonGreen, Kaede (Green), mClover3, Clover, VFP, pcDronpa2 (Green), moxNeonGreen, tdimer2(12), Dronpa (On), YPet, Skylan-NS (On), ffDronpa (On), eechGFP2, gfasGFP, Gamillus (On), sarcGFP, vsfGFP-9, mEos3.1 (Green), pmeaGFP1, mVFP, pmimGFP1, pmimGFP2, mEos4a (Green), pcDronpa2 (Red), pdaelGFP, pmeaGFP2, mScarlet, mCitrine, ccalGFP3, phiYFP, SYFP2, Citrine2, mVFP1, Gamillus0.4, SHardonnay, aacuGFP2, mVenus, mEos4b (Green), mKOÎ^{o}, fabdGFP, mGeos-C (On), rsKame (On), TurboRFP, afraGFP, stylGFP, phiYFPv, FoldingReporterGFP, Citrin, dendFP (Green), PSmOrange (Orange), anobGFP, mRuby3, RFP611, Topaz, SEYFP, mScarlet-I, mWasabi, iq-mVenus, meffRFP, d2EosFP (Green), eqFP578, EYFP-Q69K, mTFP1, ccalRFP1, eechGFP3, cgreGFP, SuperfolderGFP, meffGFP, mEos3.2 (Green), pporGFP, muGFP, Venus, mGeos-E (On), Gamillus0.2, mEosFP-M159A (Green), pporRFP, pcDronpa (Red), d1EosFP (Green), moxGFP, oxGFP, EosFP (Green), amilFP513, KO, mGeos-S (On), tdKatushka2, mOrange, mEYFP, anm1GFP1, meffCFP, AzamiGreen, obeGFP, TagRFP, dimer2, dTomato, mEos2 (Green), usGFP, M355NA, cgfTagRFP, mGeos-F (On), EYFP, Gamillus0.3, Kohinoor (On), amilFP593, ccalOFP1, obeYFP, mGeos-M (On), moxVenus, oxVenus, WasCFP, mEos4a (Red), mUkG, NowGFP, mEosFP (Green), mRuby2, ppluGFP2, mAvicFP1, dTFP0.2, AvicFP1, scubRFP, mKO2, UnaG, mEos4b (Red), GFPmut2, mRuby, Emerald, mEmerald, ppluGFP1, meleRFP, mGeos-L (On), OFP, mmi1CFP, KikGR1 (Green), TurboGFP, mApple, CyRFP1 (CyRFP1), E2-Red/Green, ccalGFP1, mPapaya, moxCerulean3, GFP(S65T), eqFP611, meleCFP, GFPmut3, SGFP2(E222Q), mCerulean3, mOrange2, G3, Dreiklang (On), TagGFP2, mAzamiGreen, mKikGR (Green), iq-mEmerald, cfSGFP2, Dendra2-M159A (Orange), mEGFP, EGFP, TagRFP-T, TagGFP, anobCFP2, KCY, td-RFP611, TagBFP, smURFP, mTagBFP2, mLumin, SGFP2, SGFP2(T65G), PSmOrange2 (Orange), eqFP611V124T, efasCFP, TagYFP, mKO, TDsmURFP, CyOFP1, mEos2 (Red), SGFP2(206A), LanFP1, rsFastLime (On), mTFP0.7 (On), cerFP505, psamCFP, Katushka2S, DsRed.T3, E2-Crimson, FR-1, DsRed2, mCerulean2, Dendra2-M159A (Green), PATagRFP1314 (On), mTurquoise2, Padron (On), aceGFP, AcGFP1, NijiFP (Orange), SPOON (on), Cerulean, amilFP490, dTFP0.1, PATagRFP1297 (On), mT-Sapphire, T-Sapphire, NijiFP (Green), mAmetrine, mNectarine, mStrawberry, SGFP1, cgfmKate2, BrUSLEE, rsGreen1 (Bright), mIrisFP (Green), G2, mTurquoise, moxDendra2 (Green), iq-mCerulean3, PATagRFP (On), mKate2, d1EosFP (Red), Turquoise-GL, MiCy, mBlueberry2, FusionRed-M, dendFP (Red), Dendra2-T69A (Green), anobCFP1, Î±GFP, mCerulean2.N, LSSmOrange, mCerulean2.D3, cpT-Sapphire174-173, Aquamarine, oxCerulean, mEosFP (Red), mEosFP-F173S (Green), KikGR1 (Red), mNeptune2.5, EBFP1.5, Dendra2-T69A (Orange), EosFP (Red), aacuGFP1, bsDronpa (On), Dendra2 (Green), mKateS158A, IrisFP (Green), ZsGreen, mCerulean.B24, obeCFP, Katushka, Padron0.9 (On), mNeptune2, AausGFP, TagCFP, mCerulean.B, LanFP2, mKateS158C, mEos3.1 (Red), Dronpa-2 (On), D10, shBFP-N158S/L173I, Kaede (Red), sg25, d2EosFP (Red), mCerulean2.N(T65S), avGFP, E2-Orange, BDFP1.6, DsRed-Max, mCerulean.B2, mIrisFP (Red), CGFP, Dendra2 (Red), Dronpa-3 (On), Sapphire, EBFP1.2, rsEGFP2 (On), FusionRed, EBFP2, CyPet, eforCP, mEos3.2 (Red), mKikGR (Red), mBeRFP, mCyRFP1, mKillerOrange, RFP630, mCherry2, moxBFP, oxBFP, KillerOrange, moxDendra2 (Red), mClavGR2 (Red), cFP484, rsEGFP (On), KCY-G4219, Gamillus0.1, mMaple (Red), SCFP3A, IrisFP (Orange), RFP637, mCardinal, mRFP1-Q66T, mKalama1, mCerulean, mKateM41GS 158C, SBFP2, KCY-R1, mPapaya0.7, mCherry, iq-EBFP2, eqFP650, G1, sg12, mMiCy, mEos2-A69T (Green), SCFP3B, DsRed-Express2, mKate, mScarlet-H, Dendra (Green), mClavGR2 (Green), td-RFP639, Azurite, Dendra (Red), miRFP670-2, PA-GFP (On), iRFP713/V256C, miRFP680, mECFP, SuperNovaRed, mNeptune, DsRed.T4, BFP.AS, mCRISPRed, ECFP, ZsYellow1, Neptune, mRaspberry, rsFolder (Green), PAmCherry2 (On), DsRed-Express, moxMaple3 (Red), iRFP670, mRFP1, mMaple3 (Red), RFP639, iq-mApple, emiRFP670, miRFP670, shBFP, SiriusGFP, AvicFP4, W7, H9, SCFP2, mRFP1-Q66S, mTangerine, IrisFP-M159A (Green), KillerRed, mMaple (Green), dsFP483, GZnP3, PS-CFP2 (Green), sg11, mRFP1-Q66C, miRFP670nano, mEosFP-F173S (Red), miRFP682, LSSmCherry1, rsFolder2 (Green), iRFP682, R3-2+PCB, amFP486, PSmOrange (Far-red), mGarnet2, miRFP, Jred, mMaroon1, PS-CFP2 (Cyan), mGarnet, zFP538, PAmCherry1 (On), miRFP670v1, W1C, dTG, rsFusionRedl (On), P4-1, emiRFP703, miRFP703, mKelly2, mStable, dKeima, mEos2-A69T (Orange), iRFP702, deGFP2, PSmOrange2 (Far-red), miRFP702, 1anRFP-Î″S831, 1aRFP, W2, mKelly1, SCFP1, miRFP713, iFP2.0, pHuji, mIFP, iFP1.4, SuperNovaGreen, HcRed-Tandem, EBFP, iRFP713, SOPP3, SOPP2, miniSOG, HcRed7, miRFP720, RDSmCherry0.1, P4-3E, mTFP0.3, mMaple3 (Green), mCarmine, iRFP720, SOPP, Maroon0.1, moxMaple3 (Green), PS-CFP (Cyan), BFP, mBlueberryl, eechRFP, PS-CFP (Green), deGFP3, PAmCherry3 (On), RDSmCherry1, deGFP1, PAmKate (On), LSS-mKate2, Wi-Phy, rsFusionRed2 (On), miRFP709, eqFP670, mBanana, KFP1 (On), sg50, mPlum, rsTagRFP (ON), deGFP4, iq-mKate2, sg42, Pp2FbFPL30M, TagRFP675, mRojoB, AQ143, Sirius, mKeima, TagRFP657, ECGFP, SNIFP, tKeima, Pp2FbFP, rsFusionRed3 (On), AsRed2, LSS-mKate1, AvicFP2 (pre-conversion), ECFPH148D, dKeima570, mHoneydew, cpCitrine, rsCherry (On), mNeptune681, mGrap1, mGinger2, mGrape3, mNeptune684, mGinger1, RDSmCherry0.2, mGrape2, mRojoA, SBFP1, mRouge, P4, RDSmCherry0.5, GZnP3 (GZnP3(apostate)), rsCherryRev (On), Sandercyanin, HcRed, mRtms5, anm2CP, mTFP1-Y67W, Ultramarine, 10B, 11, 22G, (3-F)Tyr-EGFP, 5B, 6C, Ala, A44-KR, aacuCP, AausFP2, AausFP3, AausFP4 (On), acanFP, aceGFP-G222E-Y220L, aceGFP-h, Achilles, AdRed, AdRed-C148S, ahyaCP, alajGFP1, alajGFP2, alajGFP3, amCyan1, amFP495, amFP506, amFP515, ami1CP, ami1CP580, amilCP586, amilCP604, amilFP484, amilFP497, amilFP504, amilFP512, amilFP597, anm1GFP2, apulCP584, apulFP483, AQ14, asCP562, asFP499, asulCP, atenFP, avGFP454, avGFP480, avGFP509, avGFP510, avGFP514, avGFP523, AvicFP3 (pre-conversion), bfloGFPc1, BFP5, BFPso1, Blue102, BR1, cEGFP, CFP, CFP4, cgigCP, cgigGFP, CheGFP1, CheGFP2, CheGFP3, CheGFP4, Clomeleon, Clover1.5, cpasCP, cp-mKate, Cy11.5, dClavGR1.6, dClover2, dClover2A206K, dfGFP, dhorGFP, dhorRFP, dimer1, dis2RFP, dis3GFP, dPapaya0.1, DrCBD, d-RFP618, Dronpa-C62S, DspR1, DsRed.M1, DsRed-Timer, DstC1, EaGFP, echFP, echiFP, eGFP203C, eGFP205C, EnhancedCyan-EmittingGFP, EYFP-F46L, fcFP, fcomFP, Flamindo2, FP586, Fpaagar, Fpag_frag, Fpcondchrom, FPmann, FPmcavgr7.7, FPrfl2.3, Gamillus0.5, GCaMP2, GCaMP6f (in presence of Ca²⁺), gdjiCP, gfasCP, GFP-151pyTyrCu, GFPhal, GFP-Tyr151pyz, GFPxm16, GFPxm161, GFPxm162, GFPxm163, GFPxm18, GFPxm181uv, GFPxm18uv, GFPxm19, GFPxm191uv, GFPxm19uv, gtenCP, HcRed1-Blue, hcriCP, hcriGFP, hfriFP, hmGFP, HriCFP, HriGFP, iLov, jRGECO1a (in absence of Ca²⁺), jRGECO1a (in presence of Ca²⁺), Katushka-9-5, KCY-G4219-38L, KCY-R1-158A, KCY-R1-38H, KCY-R1-38L, KikG, KOFP-7 (KOFP-7), laesGFP, laGFP, LEA, mc1, mc2, mc3, mc4, mc5, mc6, McaG1, McaG1ea, McaG2, mcavFP, mcavGFP, mcavRFP, mcCFP, mcFP497, mcFP503, mcFP506, mCherry1.5, mClavGR1, mClavGR1.1, mClavGR1.8, mClover1.5, mcRFP, meffCP, mEos2-NA, meruFP, MfaG1, miniSOG2, miniSOGQ103V, mKate2.5, mKG, mK-GO (Late), mK-GO (Early), mMaple2 (Green), mMaple2 (Red), mmGFP, mOFP.T.12, mOFP.T.8, montFP, Montiporasp.#20-9115, moxEos3.2, mPA-GFP, mPapaya0.3, mPapaya0.6, mPlum-E16P, mRed7, mRed7Q1, mRed7Q1S1, mRed7Q1S1BM, mRFP1.1, mRFP1.2, mRFP1.3, mRFP1.4, mRFP1.5, mTFP*, mTFP0.4, mTFP0.5, mTFP0.6, mTFP0.8, mTFP0.9, mTFP1-Y67H, mTurquoise-146G, mTurquoise-146S, mTurquoise2-G, mTurquoise-DR, mTurquoise-GL, mTurquoise-GV, mTurquoise-RA, NpR3784g, OFPxm, P11, P9, Padron(star) (On), PdaC1, PDM1-4, pHluorin2 (acidic), pHluorin2 (alkaline), pHluorin, psupFP, pti1GFP, Q80R, RCaMP, R-FlincA, rfloGFP, rfloGFP2, rfloRFP, RFP618, roGFP1, roGFP1-R1, roGFP1-R8, roGFP2, RpBphP1, RpBphP2, RpBphP6, rrenGFP, rrGFP, rsCherryRev1.4 (On), RSGFP1, RSGFP2, RSGFP3, RSGFP4, RSGFP6, RSGFP7, Rtms5, SAASoti (Red), SAASoti (Green), scleFP1, scleFP2, scubGFP1, scubGFP2, secBFP2, sfCherry, sfCherry2, sfCherry3C, SH3, ShG24, spisCP, stylCP, SuperfoldermTurquoise2, SuperfoldermTurquoise2ox, sympFP, TeAPCÎ±, tPapaya0.01, Trp-lessGFP, TurboGFP-V197L, V127TSAASoti (Red), V127TSAASoti (Green), vsGFP, Xpa, yEGFP, YFP3, zGFP, zoan2RFP, zRFP, or any other fluorescent derivative thereof. The fluorescent proteins are genetically encoded.

According to an embodiment, the link between the fluorochrome Fₐ and the receptor is a covalent bond. In a specific configuration of this embodiment, this bond is of the type NHS-NH₂, maleimide-SH or derivatives thereof, the NHS or maleimide group being carried on the fluorochrome Fₐ and the amine or thiol being on the receptor.

According to an embodiment wherein the fluorochrome Fₐ is a fluorescent protein, the receptor and Fₐ are bonded in fusion protein form. In this embodiment, the receptor and Fₐ are coded by the same gene.

According to an embodiment, the link between the fluorochrome F_{b} and the polypeptide is a covalent bond. In a specific configuration of this embodiment, this bond is of the type NHS-NH₂, maleimide-SH or derivatives thereof, the NHS or maleimide group being carried on the fluorochrome F_{b} and the amine or thiol being on the polypeptide.

According to an embodiment, in the inactive configuration of the fluorescent probe (OFF configuration), the distance between the fluorochromes Fₐ and F_{b} does not enable the FRET effect, i.e. the distance between the fluorochromes Fₐ and F_{b} is greater or less than the distance enabling the FRET effect; whereas in the active configuration (ON configuration), the distance between the fluorochromes Fₐ and F_{b} enables the FRET effect. In other words, the distance between the fluorochromes Fₐ and F_{b} in the inactive configuration (i.e. fluorescent probe at rest, OFF configuration) is greater or less than the distance between the fluorophores Fₐ and F_{b} in the active configuration (ON configuration). The fluorescent probe is at rest when the receptor is not bonded to a target molecule; in the case where the receptor is an antibody, the fluorescent probe is at rest when the antibody recognition site is free. The fluorescent probe is in the active configuration when the receptor is bonded to a target molecule; in the case where the receptor is an antibody, the fluorescent probe is in the active configuration when the antibody recognition site is not free, i.e. an antigen is recognised and bonded to the antibody. There is also a scenario where the ON configuration occurs when the antibody does not detect the target molecule thereof and which switches to the OFF configuration when the receptor is bonded to the target molecule (FRET without target molecule and stopping of FRET with). This results in both cases to a modification of the fluorescent signal on the recognition of a target molecule.

Device for Detecting a Target Molecule and/or Measuring the Concentration of a Target Molecule

The present invention also relates to a device for detecting a target molecule and/or measuring the concentration of a target molecule.

The device comprises:
a substrate at the surface of which a grafting molecule is covalently attached;
at least one fluorescent probe comprising:
   at least one receptor bonded to a polypeptide via a covalent bond;
   two fluorochromes Fₐ and F_{b};
wherein the fluorochrome Fₐ is bonded to the receptor and the fluorochrome F_{b} is bonded to the polypeptide; and
the fluorochromes Fₐ and F_{b} form a FRET donor/acceptor pair.

The polypeptide is bonded to the grafting molecule via a covalent bond.

The fluorescent probe is as described above such that the embodiments relating to the fluorescent probe or the different elements of said probe (receptor, polypeptide, fluorochromes) apply to the device of the invention.

A covalent bond between the receptor and the polypeptide, stronger than the receptor-target molecule bond, and a covalent bond between the polypeptide and the grafting molecule makes it possible to ensure that, once the target molecule has been recognised, the receptor will not be detected from the polypeptide or that the fluorescent probe will not be detached from the substrate. Thus the separation of the fluorescent probe into two parts or probe-substrate separation is avoided.

During an in vitro target molecule detection, avoiding the separation between the receptor and the polypeptide is particularly important so as not to induce a contamination of the sample.

During an in vivo target molecule detection, avoiding the separation between the receptor and the polypeptide or the separation between the fluorescent probe and the substrate is particularly important, particularly when the fluorescent probe is used at the distal end of an optical fibre with a view to intracorporeal exploration, as this limits the risk of leaving a part (that with the receptor) or all of the fluorescent probe in the patient's body when removing the fibre. Such a biological contamination can have various adverse effects, similar to those observed during direct antibody injection, such as for example signs of discomfort such as headaches, nausea or feeling of weakness, reactions such as fever or shivers, allergic-like, cutaneous tropism (pruritus, eruption, urticaria), respiratory (bronchospasms, cough, dyspnoea) or cardiovascular (hypotension) symptoms, or tumour lysis syndromes (in cases of substantial tumour mass). These adverse effects are due both to the nature of the ligand and that of the receptor.

The role of the grafting molecule is that of ensuring the correct orientation of the receptor so that the recognition sites thereof are accessible to the target molecule.

The use of a grafting molecule on the surface of the substrate also makes it possible to finely control the functionalisation of said surface, particularly modulate the number of fluorescent probes grafted in the surface of the substrate, i.e. finely control the fluorescent probe density per unit of surface of the substrate (or coverage ratio), by modifying the coverage of said substrate by the grafting molecule. This control advantageously makes it possible to provide a device intended for the user's specific need.

In an alternative aspect of the invention, the polypeptide could be bonded to the grafting molecule via a non-covalent bond.

According to an embodiment, the target molecule (or ligand) is a molecule for which the receptor has an affinity and a strong specificity. Preferably, the target molecule (or ligand) is an antigen.

According to an embodiment, the substrate is chosen from a cell culture plate, a well plate, a film, a strip, an agarose gel, a cellulose gel, nanoparticles or microparticles, preferably spherical, preferably silica or polymer, a microscope slide, or a glass strip. The purpose of a device according to this embodiment is the detection and/or measurement of the concentration of target molecules in vitro, i.e., in solution or on substrate.

Preferably, the substrate is a polymer film. Said polymer film is chosen from polyethylene terephthalate, fluorinated polyethylene-co-propylene, polymethylmethacrylate, polytetrafluoroethylene, polymethylpenthene, polyvinyl chloride, styrene methyl methacrylate, polyethylene naphthalate, derivative thereof or a mixture thereof. Advantageously, such a polymer film is chemically inert, transparent, and/or resistant to high temperature, i.e., resistant to a temperature of at least 90° C., preferably at least 110° C., preferably at least 130° C.

According to an embodiment, the device further comprises an optical fibre. Preferably, the device comprises an optical fibre and an exploration head. The head being attached removably or rigidly connected to the optical fibre (via a ferrule). In a known manner, an optical fibre comprises a cladding encasing one or more fibre cores, and the distal end thereof intended for the exploration is presented in the form of a rigid ferrule rendering firmly rigidly connected to the end of the fibre cladding and wherein the outer face transversal to the fibre axis is transparent. The purpose of a device according to this embodiment is the detection and/or measurement of the concentration of target molecules in vivo, i.e., in the patient's body for example by endoscopy or during surgery.

The “exploration head” is the part of the fibre acting as a probe or as a general rule having any technical function using light emitted at the end of the optical fibre to cooperate or interact with the medium wherein the end of the fibre is introduced. The ferrule of the optical fibre only has a mechanical attachment function for the exploration head.

In the case of a removable exploration head, the ferrule and the exploration head are rigidly connected by mechanical assembly (crimping, fitting, screwing, clipping, quarter-turn locking), or respectively comprise mutual cooperation attachment means (male-female cooperation means). The attachment of the head to the ferrule is designed such that the head and the ferrule cannot be detached during use, in particular when the fibre has been introduced into the patient's body.

In a specific configuration of this embodiment, the substrate is chosen from the periphery of an optical fibre or a substrate configured to be attached to the head of an optical fibre, preferably an optically transparent substrate configured to be attached to the distal end of an optical fibre (i.e. the exploration end), more specifically, to the distal end of the exploration head.

Preferably, the substrate is a polymer film located at and/or attached to the distal end of the exploration head. In this embodiment, the polymer film can be glued to the distal end of the exploration head, with for example an epoxy resin, or mechanically attached to the distal end of the exploration head.

In a specific configuration of this embodiment, the exploration head has a body and an outer face at the distal end thereof, referred to as emission face, at least a part of which is transparent forming a port, and intended to be facing the core(s) of the optical fibre for the passage of light. Preferably, a polymer film is used as a transparent part such that it is functionalised with the fluorescent probe, i.e. the substrate of the device is the port of the exploration head. This advantageously makes it possible to conduct a corporeal exploration and an in vivo target molecule detection by placing the distal end of the exploration head in contact with a member. Preferably, the exploration head is attached removably to the optical fibre, advantageously making it possible to change exploration head and therefore fluorescent probe according to the target molecule detection while retaining the same optical fibre. Preferably, the body is made of polymeric material, glass, ceramic, stainless steel, composite material, or combination of these materials, and the outer emission face is made of polymer, glass, ceramic, silica, composite or hybrid material.

Advantageously, the optical fibre does not undergo any treatment such as tapering. Preferably, the fluorescent probe is grafted on the port of an exploration head, therefore the fibre remains intact. This makes it possible to improve the sensitivity of the device and the reproducibility of the grafting surface. The role of the optical fibre is then that of transporting light signal to the target.

According to an embodiment wherein the optical fibre comprises a core partially covered by a metallic cladding, a portion of the core not being covered by the cladding, the fluorescent probe is grafted on the surface of the portion of the uncovered core. This embodiment makes it possible to obtain a surface wave on the longitudinal part of the optical fibre.

According to an embodiment, the grafting molecule comprises at least two reactive groups chosen from maleimide, N-Hydroxysuccinimide (NHS) ester, sulfo N-hydroxysuccinimide ( NHS) ester, sulfo-NHS, azide, alkyne, epoxide, carboxylic acid, aldehyde, aziridine, alkene, or a derivative thereof. The grafting molecule enables the covalent link between the substrate and the fluorescent probe via grafting molecule-polypeptide bond. Preferably, the two reactive groups are located at each of the ends thereof. The bond between the polypeptide and the grafting molecule takes place between a terminal group of the polypeptide, preferably thiol or amine, and one of the two reactive groups of the grafting molecule described here.

In a preferred configuration of this embodiment, the grafting molecule comprises a maleimide group capable of reacting with a thiol terminal group of the polypeptide to form a covalent bond. The thiol group being terminal and single, this configuration has the advantage of being able to control the orientation of the polypeptide on the substrate, hence that of the fluorescent probe, making it possible to ensure better accessibility of the receptor for a target molecule.

In another configuration of this embodiment, the grafting molecule comprises an N-hydroxysuccinimide group capable of reacting with an amine group of the polypeptide to form a covalent bond.

According to an embodiment, the substrate is covered with a layer of organic or inorganic material chosen from zirconia, titanium dioxide, epoxy, organosilane such as for example amine organosilane, thiol organosilane, azide organosilane, alkyne organosilane, carbonyl organosilane, or organosilane having a double carbon-carbon bond. Preferably, the substrate is covered with a layer of amine organosilane or thiol organosilane. Typically, the substrate, for example a polymer film, is covered with an amine organosilane then steeped in a (3-Aminopropyl)triethoxysilane (APTES) solution resulting in a substrate covered with a layer of grafting molecule carrying a maleimide group. Alternatively, the substrate, for example a polymer film, is covered with a thiol organosilane then steeped in an amine solution resulting in a substrate covered with a layer of grafting molecule carrying a maleimide group.

Method for Detecting a Target Molecule and/or Measuring the Concentration of a Target Molecule.

The present invention also relates to a method for detecting a target molecule and/or measuring the concentration of a target molecule.

The method comprises the following steps:
Placing a sample and at least one fluorescent probe in contact, said fluorescent probe comprising:
   at least one receptor bonded to a polypeptide via a covalent bond;
   two fluorochromes Fₐ and F_{b};
wherein the fluorochrome Fₐ is bonded to the receptor and the fluorochrome F_{b} is bonded to the polypeptide; and
the fluorochromes Fₐ and F_{b} form a FRET donor/acceptor pair; and the receptor has an affinity for said target molecule;
Exciting the fluorescent probe at a given wavelength such that the donor fluorochrome is excited;
Measuring the ratio between the intensity of the fluorescence emitted by the donor fluorochrome and the intensity of the fluorescence emitted by the acceptor fluorochrome;
Determining the presence or the absence of said target molecule in the sample and/or calculating the concentration of said target molecule in the sample.

On the recognition of a target molecule by the receptor, a conformational change of said receptor takes place, inducing a variation of the distance between the fluorochromes Fₐ and F_{b}, resulting in a non-radiative transfer of the donor fluorochrome to the acceptor fluorochrome (FRET effect). The variations in the intensity of the fluorescence peaks of the donor and acceptor fluorochromes are thus the direct consequence of the target molecule concentration in the sample. An increase or a decrease of the fluorescent intensity emitted by the donor fluorochrome is therefore expected if the target molecule is present in the sample, this results in an increase or a decrease in the ratio between the intensity of the fluorescence emitted by the donor fluorochrome and the intensity of the fluorescence emitted by the acceptor (hereinafter referred to as FRET index). If the intensity of the fluorescence peaks remains unchanged (zero FRET index), this indicates the absence of target molecule in the sample. Thus, determining the presence or the absence of said target molecule in the sample and/or calculating the concentration of said target molecule in the sample results from the interpretation of the variation of the FRET index. The opposite is also possible.

The detection of the target molecule is advantageously rapid. The use of the fluorescent probe as described above, particularly the presence of a covalent receptor-polypeptide bond, gives rise to a significant increase in the target molecule detection sensitivity. For example, it was observed that the detection threshold was less than 25 nM. Preferably, the method according to the invention has a detection threshold less than 10 nM, preferably less than 5 nM, more preferably less than 1 nM, even more preferably less than 0.1 nM.

The embodiments relating to the fluorescent probe, the different elements of said probe (receptor, polypeptide, fluorochromes), the device or the different elements of said device apply to the implementation of the method according to the invention. In particular, the method according to the invention is implemented by the device according to the invention.

According to an embodiment, the fluorescent reporter is placed in contact with the sample by any contacting means. Preferably, the contacting takes place in solution or by direct touch.

In a specific configuration of this embodiment, the contacting by direct touch lasts for a few seconds. In another specific configuration of this embodiment, for example in solution, the contacting in solution lasts for less than one hour, preferably less than 30 minutes, more preferably less than 5 minutes. The greater the contacting time, the sharper the optical signal.

According to an embodiment, the ratio between the intensity of the fluorescence emitted by the donor fluorochrome and the intensity of the fluorescence emitted by the acceptor fluorochrome is measured by spectrometry.

According to an embodiment, the method also comprises a prior calibration step whereby the device is placed into contact with a healthy sample. Advantageously, this step makes it possible to determine a reference FRET index measurement for the chosen Fₐ/F_{b} pair. Subsequently, the comparison between this reference measurement and the FRET index measured in contact with the sample suspected of carrying the target molecule will enable a conclusion on the presence or not of the target molecule as well as the concentration thereof in the tested sample.

The presence, absence, or detection of a quantity of target molecule above or below a certain threshold will make it possible to characterise the sample, and consider it healthy or not.

According to an embodiment, the sample can be any sample having the possibility of containing a target molecule as a detection or measurement object and can be a liquid sample or a solid sample.

According to an embodiment, the sample is chosen from a solution, a cell culture (for example eukaryote or prokaryote), whole blood, plasma, blood serum, sweat, or any biological liquid or fluid, an organic tissue, or an organ.

In a specific configuration of this embodiment, a liquid sample can be directly used as a detection or measurement object or can be diluted with, for example, a buffer solution, a saline solution, then can be used as a detection or measurement object. Examples of the liquid sample comprise but are not limited to a cell culture (for example eukaryote or prokaryote), culture supernatants, cell extracts, bacterial extracts, bodily fluids such as, for example, serum, plasma, saliva, sweat, cerebrospinal fluid or urine, industrial wastewater, or an agri-food liquid such as, for example, milk.

In a specific configuration of this embodiment, a solid sample is chosen from an organic tissue, an organ. The solid sample can be dissolved, suspended or submerged in a liquid, such as a buffer solution or a saline solution, in a state capable of being in contact with the free fluorescent probe and is then used as a sample. Preferably, the solid sample does not undergo any treatment before placing in contact with the fluorescent probe.

According to an embodiment, the detection threshold of the target molecule is dependent on the affinity of the receptor with the target molecule, the detection threshold is of the order of a few pmol·L⁻¹ (picomolar), preferably of the order of a few fmol·L⁻¹ (femtomolar).

According to an embodiment, the method can be implemented:
In vitro, for example in suspension, in solution, or on a substrate (e.g. multi-well plate, microscope slide or strip); or
In vivo, for example by endoscopy, or during surgery.

According to an embodiment, the device also comprises an excitation means configured to excite the sample and/or the fluorescent probe, and/or an optical collection means configured to collect the data from the fluorescence of the fluorochromes Fₐ and F_{b}.

In a specific configuration of this embodiment, the excitation means is a light source capable of performing irradiation with a given wavelength such as, for example a mercury lamp, a xenon lamp, a LED, a UV lamp or a laser light source.

In a specific configuration of this embodiment, the optical data collection means is a microscope, a fluorimeter, a cytometer, or a spectrophotometer.

### Uses

The present invention also relates to the use of the fluorescent probe according to the invention and/or of the device according to the invention for detecting a target molecule and/or measuring the concentration of a target molecule in a sample.

According to an embodiment, the fluorescent probe according to the invention and/or the device according to the invention are used for detecting a target molecule and/or measuring the concentration of a target molecule in a sample in vitro, for example in solution.

According to an embodiment, the fluorescent probe according to the invention and/or the device according to the invention are used for detecting a target molecule and/or measuring the concentration of a target molecule in a sample in vivo.

In a specific configuration of the in vivo embodiment, the fluorescent probe according to the invention and/or the device according to the invention are used for detecting tumour cells, screening for infection, or detecting markers capable of assisting with the diagnosis or monitoring of the progression of a condition. In a specific configuration of the in vivo embodiment, the device is an optical fibre comprising an exploration head to which is grafted the fluorescent probe (in this case, the fluorescent probe is grafted on the port of the exploration head, i.e. the transparent part of the emission face of said head), or a catheter at the distal end of which the fluorescent probe is grafted. Preferably, the fluorescent probe according to the invention and/or the device according to the invention are used in endoscopy. The endoscopy is used to examine the interior of a cavity by a backscattered light detection technique thanks to an optical fibre. An endoscope comprises a flexible casing housing one or more optical fibres the distal end of which is intended to be introduced into the cavity to be examined, and the opposite proximal end is intended to be connected to a light source aligned with the optical fibre(s) to transmit light to the distal end and in the cavity, light detectors disposed also in alignment with the optical fibres and at the proximal end being intended to receive the light emitted by the fluorescent probe and transmitted via the fibres in return.

According to an embodiment, the fluorescent probe according to the invention and/or the device according to the invention are used for detecting a target molecule and/or measuring the concentration of a target molecule in industrial water, wastewater or an agri-food liquid such as milk for example. In a specific configuration of this embodiment, the fluorescent probe according to the invention and/or the device according to the invention are used for detecting medicinal product or pesticide residue, or detecting pathogens.

### DESCRIPTION OF THE FIGURES

FIG. 1 is a diagram representing the switch of the fluorescent probe from an inactive configuration (off) to an active configuration (on) on the recognition of a target molecule.

FIG. 2 represents a detection device according to a specific embodiment.

FIG. 3A is an illustration of the optical spectra obtained by fluorimetry analysis of the response of the fluorescent probe to different antigen concentrations.

FIG. 3B is an illustration of the dose-response curve obtained with the fluorescent probe, by measuring the FRET indices using the curves shown in FIG. 3A.

FIG. 4 is illustration of the FRET response obtained on adding an antigen to the fluorescent probe (here TrkB) and a non-relevant molecule, BSA where the conformational change takes place.

FIG. 5A is an illustration of the optical spectra measured after exposure of the fluorescent probe with a 488 nm laser in the present of cell lines expressing the target antigen or not.

FIG. 5B is an illustration of the FRET indices obtained from the spectra shown in FIG. 5A.

FIG. 6 is an illustration of the optical spectra measured after excitation with a 488 nm laser of a fluorescent probe comprising an antibody bonded to a protein G (grey curve) and a fluorescent probe comprising an antibody bonded to a polypeptide consisting of 8 amino acids, of which the amino acid sequence as described in SEQ ID NO:1 (black curve).

FIG. 7 is an illustration of the fluorescence intensity after excitation with a 488 nm laser of a fluorescent probe comprising an antibody bonded to a binding protein non-covalently (S_{nc}) and of a fluorescent probe comprising an antibody bonded to a binding protein covalently (S_{c}) before or after eluting the antibody by acid pH (white column: solution containing the fluorescent probe before elution, grey column: solution containing the fluorescent probe after elution, black column: eluent).

FIG. 8 is an illustration of the amplitude of the change of FRET index of the fluorescent probes in response to exposure to the target antigen EGFR in the case of a fluorescent probe comprising an antibody bonded to a binding protein non-covalently (S_{nc}) and a fluorescent probe comprising an antibody bonded to a binding protein covalently (S_{c}).

### ILLUSTRATIVE EMBODIMENTS OF THE INVENTION

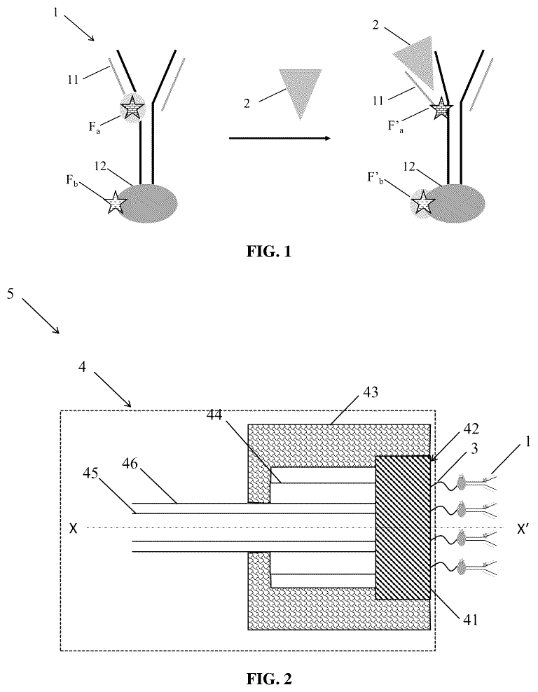

The recognition of a target molecule 2 by the fluorescent probe 1 is illustrated in FIG. 1, the fluorescent probe 1 comprises:
a receptor 11 bonded to a polypeptide 12; and
two fluorochromes Fₐ and F_{b}.

The fluorochrome Fₐ is bonded to the receptor 11 and the fluorochrome F_{b} is bonded to the polypeptide 12. The fluorochromes Fₐ and F_{b} form a FRET donor/acceptor pair. The receptor 11 is bonded to the polypeptide 12 via a covalent bond, i.e., stronger than that capable of bonding the receptor 11 to a recognition molecule 2.

Before the recognition of the target molecule 2 by the fluorescent probe 1, the latter is in a so-called inactive configuration (“OFF”), i.e., there is no FRET effect between the two fluorochromes Fₐ and F_{b}. In this configuration, the donor fluorochrome emits light by fluorescence as it is excited whereas the acceptor fluorochrome does not emit any. On the recognition of the target molecule 2 by the fluorescent probe 1, the latter then takes an active configuration (“ON”), a conformational change of the receptor 11 takes place, modifying the distance which separates the two fluorochromes Fₐ and F_{b}, thus inducing a non-radiative energy transfer (FRET effect) between the two fluorochromes. The energy transfer takes place from the donor fluorochrome to the acceptor fluorochrome: the intensity of the fluorescence of the donor fluorochrome decreases, and that of the acceptor fluorochrome increases; in this configuration, the fluorochromes are annotated F′ₐ and F′_{b}. The variation of the emission spectrum thereof due to the FRET effect can be measured to detect and/or measure the concentration of target molecule 2.

This embodiment is particularly advantageous as it enables a rapid detection of the target molecule 2 while avoiding the degradation of the fluorescent probe 1 as the receptor 11-polypeptide 12 bond prevails over the receptor 11-target molecule 2 bond.

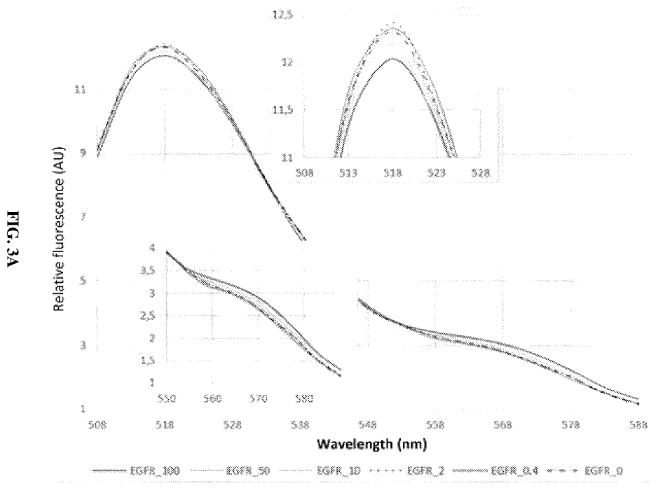

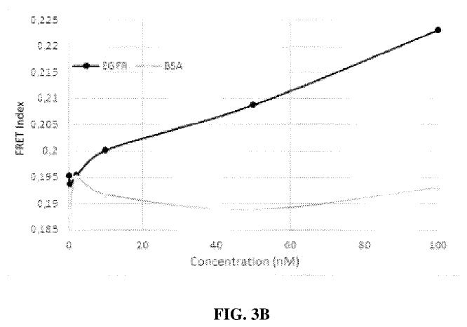

In an embodiment illustrated in FIG. 2, the device for detecting a target molecule comprises:
an optical fibre 4 comprising:
   a cladding 46;
   at least one core 45 of longitudinal axis XX′;
   a ferrule 44 ▪ an exploration head comprising a body 43 and an outer face 42, referred to as emission face, at least a part of which is transparent forming a port 41, and intended to be facing the core(s) 45 of the optical fibre 4 for the passage of light; and
a fluorescent probe 1 comprising:
   a receptor bonded to a polypeptide; and
   two fluorochromes Fₐ and F_{b}; the fluorochrome Fₐ is bonded to the receptor 11 and the fluorochrome F_{b} is bonded to the polypeptide 12, the fluorochromes Fₐ and F_{b} form a FRET donor/acceptor pair.

The optical fibre 4 has a proximal end, not illustrated here, which is intended to be connected in a known manner to a light source, and a distal opposite end, forming the exploration end of the optical fibre 4 from which the light required to illuminate the fluorescent probe will be emitted. The same optical fibre is also used for collecting the light response of the probe and leads the return light beam to a box at the proximal end of the fibre.

The exploration end of the optical fibre 4 is provided in a known manner with a ferrule 44 forming a rigid endpiece perforated at the centre thereof and wherein the cladding 46 of the optical fibre 4 is attached.

The port 41 on the outer face 42 of the exploration head is functionalised by the fluorescent probe 1 via a grafting molecule 3. The fluorescent probe 1 can thus recognise a target molecule present in the cavity explored by the optical fibre 4, particularly a target molecule encountered by the exploration head during the use thereof, as housed in a cavity of the human body, i.e., on a human tissue, for detecting cancer cells by observing and/or measuring variations in the emission spectra of fluorochromes Fₐ and F_{b}.

In a preferential manner illustrated in FIG. 2, the exploration head comprises a hollow cylindrical body 43 of the same longitudinal medial axis XX′ in the assembled position of the head on the body of the optical fibre 4, and an outer distal end face 42 which is transversal to the axis of the cylindrical body and from which light from the core 45 of the optical fibre 4 is intended to be emitted.

This embodiment is particularly advantageous as it enables a rapid detection of the target molecule in a cavity of the human body (for example by endoscopy or during surgery) while avoiding the degradation of the fluorescent probe 1 on the recognition of the target molecule. This particularly avoids leaving portions of said fluorescent probe 1 in the cavity to be explored, which would cause a contamination in the explored human body.

### EXAMPLES

The present invention will be understood more clearly on reading the following examples which illustrate the invention non-restrictively.

### Example 1a: In Vitro Target Molecule Detection—Detection in Solution

### Fluorescent Probe Preparation

A solution of anti-EGFR antibody (labelled with Alexa488) and protein G (labelled with Alexa546) in equimolar concentration is incubated for 2 h. The incubation must be long enough to ensure the optimal binding protein-antibody bond.

### Target Molecule Detection

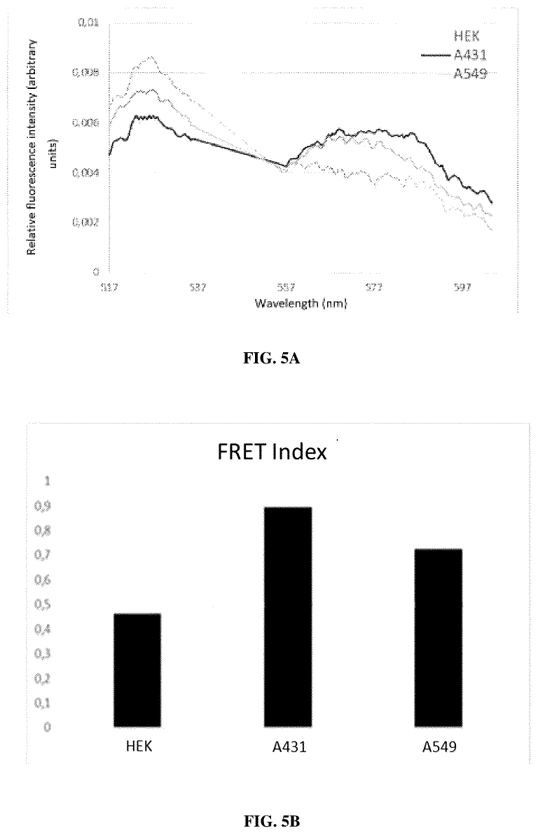

In parallel, solutions of known antigen concentrations are diluted in the same probe solution, making it possible to produce a standard series from 100 nM to 80 pM. The solution containing the antigen (recombinant EGFR) diluted in PBS is mixed with the solution containing the fluorescent probe prepared previously, to attain identical concentrations to the standard series. After incubating for one hour, the spectral properties are measured by fluorimetry. The FRET indices are calculated for the standard series and the sample, thus making it possible to calculate the concentration thereof. The results obtained for the standard curve are shown in FIG. 5A and 5B.

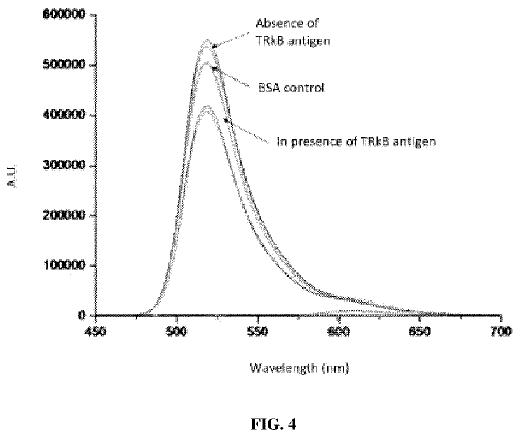

The results shown in FIGS. 4, 5A and 5B were obtained by depositing cells from lines expressing the target EGFR of the antibody or not (HEK cells, A549 cells, A431 cells), on a microscope slide whereon the fluorescent probe was previously mounted. For this, an organosilica (thiol silica)/zirconia sol is prepared 24 hours before depositing on the substrate: 20 mL of mercaptotriethoxysilane/zirconium chloride/ethanol/water mixture (0.95/0.05/40/5) is stirred at ambient temperature. The sol is deposited on a microscope slide by dip-coating (at a withdrawal rate of 5.5 mm·s⁻¹), and is heat-treated at 80° C. for 12 hours. A first binding molecule, 1-(2-amino-ethyl)-pyrrole-2,5-dione hydrochloride, containing a maleimide group and an amine group is dissolved in DMSO at 0.1 moL·L⁻¹ then this solution is added onto the slide for 1 hour under stirring at ambient temperature. After rinses and washes with DMSO and with distilled water, a second solution containing another binding molecule, N-succinimidyl 4-maleimidobutyrate, containing an NHS ester group and a maleimide in DMSO (at 0.1 moL·L⁻¹) is added onto the slide. After rinses and washes with DMSO and with distilled water, a solution of protein G (Alexa546) at 10 μg/ml in PBS is incubated on the slide for one hour. The excess is washed with PBS, then a solution of anti-EGFR antibody (Alexa488) in PBS is incubated for one hour. The excess is washed with PBS, then the A431, HEK and A549 cell suspensions are deposited on the surfaces thus functionalised.

The excitation by the laser and the collection of the signal are carried out using an optical fibre and a spectrophotometer. The results make it possible to determine the quantity of target molecule present on the surface of the cells (low to zero in HEK cells, moderate in A549 cells, high in A431 cells).

### Example 1b: In Vitro Target Molecule Detection

Example la was reproduced but the fluorescent probe was modified according to Table I.

TABLE I
Fluorescent probe compositions

| | | Binding | | | Ligand |
|---|---|---|---|---|---|
| Receptor | Fₐ | protein | F_{b} | Ligand | detection |
| Anti-TrkB | Alexa 488 | Protein G | Alexa 546 | Recombinant | yes |
| antibody | | | | TrkB | |
| Anti-TrkB | Alexa 488 | Protein G | Alexa 594 | Recombinant | yes |
| antibody | | | | TrkB | |
| Anti-EGFR | Alexa 488 | Protein G | Alexa 546 | Recombinant | yes |
| antibody | | | | EGFR | |
| (AY3 clone) | | | | | |
| Anti-EGFR | Alexa 488 | Protein G | Alexa 546 | Recombinant | yes |
| antibody (MA- | | | | EGFR | |
| 12693 clone) | | | | | |
| Anti-EGFR | CF Dye 503R | Protein G | CF Dye 555 | A431 line | yes |
| antibody (MA- | | | | cells | |
| 12693 clone) | | | | | |
| Anti-EGFR | CF Dye 503R | Protein G | CF Dye 594 | A431 line | yes |
| antibody (MA- | | | | cells | |
| 12693 clone) | | | | | |
| Anti-EGFR | CF Dye 503R | Protein G | CF Dye 555 | A549 line | yes |
| antibody (MA- | | | | cells | |
| 12693 clone) | | | | | |
| Anti-EGFR | CF Dye 503R | Protein G | CF Dye 594 | A549 line | yes |
| antibody (MA- | | | | cells | |
| 12693 clone) | | | | | |

### Example 2: In Vitro Target Molecule Detection

### Grafting on Substrate

An organosilica (thiol silica)/zirconia sol is prepared 24 hours before depositing on the substrate: 20 mL of mercaptotriethoxysilane/zirconium chloride/ethanol/water mixture (0.95/0.05/40/5) is stirred at ambient temperature. The sol is deposited on a substrate, typically the well of a multi-well plate, and is heat-treated at 80° C. for 12 hours. A first binding molecule, 1-(2-amino-ethyl)-pyrrole-2,5-dione hydrochloride, containing a maleimide group and an amine group is dissolved in DMSO at 0.1 mol·L⁻¹ then this solution is added into the well for 1 hour under stirring at ambient temperature. After rinses and washes with water and with DMSO, a second solution containing another binding molecule, N-succinimidyl 4-maleimidobutyrate, containing an NHS ester group and a maleimide in DMSO (at 0.1 mol·L⁻¹) is added into the well, 100 μL per well for lh under stirring. After rinses and washes with DMSO and with distilled water, the substrate is prepared to bind the first fluorescent binding-protein (conventionally a protein G ending with a cysteine).

### Fluorescent Probe Preparation

A solution of protein G (labelled with Alexa546) at 10 μg/ml in PBS is incubated on the functionalised surface for 1 hour. The excess is washed with PBS. A 2 mM gluteraldehyde solution is incubated with protein G immobilised on the substrate for 15 minutes. The excess glutaraldehyde is washed with PBS, then a solution of anti-EGFR antibody (labelled with Alexa488) is incubated for 2 h. This time is long enough to ensure the optimal protein G-antibody bond. The excess antibody is then washed with PBS.

### Target Molecule Detection

A solution containing the EGFR target antigen is added into the wall and will be detected in the same way as during the test in solution of example 1.

### Example 3a: In Vivo Target Molecule Detection

### Grafting on an Exploration Head or on an Optical Fibre

A fluorescent probe prepared according to the method defined above is grafted either directly onto a transparent port of an exploration head (or capsule) configured to be assembled with an optical fibre. The port typically consists of a PET (polyethylene terephthalate) or FEP (fluorinated polyethylene—co-propylene). Grafting is performed in the same way in during example 2. This means firstly via a step of thin silica/zirconia layer deposition on the port by dip-coating the organosilane/zirconia sol at a rate of 5.5 mm·s⁻¹ then via a step of functionalising the surface by the grafting molecules containing maleimide/NHS groups (1-(2-amino-ethyl)-pyrrole-2,5-dione hydrochloride, followed by N-succinimidyl 4-maleimidobutyrate at 0.1 mol·L⁻¹). The maleimide group can react with a thiol group of the protein and the NHS groups can react with an amine group of the protein to result in a covalent bond between the fluorescent probe and the substrate.

The method for grafting the fluorescent probe on the end of the fibre or on the port is comparable to the method of grafting the substrate of example 2.

### EGFR Detection on a Solid Surface

The port or the optical fibre are placed in contact with a tissue or any other interface on the surface whereof the target molecule is liable to be found and, at the same time, an illumination by laser beam at 488 nm is conveyed by the optical fibre to the fluorescent probe. The reflected beam is collected by the same optical fibre to a spectrophotometer for analysis.

The FRET index is calculated in order to determine whether the interaction between the fibre (or the port) and the tissue is positive (presence of the target molecule) or negative (absence of the target molecule). This detection is carried out in a time interval of a few seconds

### Example 3b: Target Molecule Detection

Example 3a was reproduced but the fluorescent probe was modified according to the Table II.

TABLE II
Fluorescent probe compositions

| | | Binding | | | Ligand |
|---|---|---|---|---|---|
| Receptor | Fₐ | protein | F_{b} | Ligand | detection |
| Anti-TrkB | Alexa 488 | Protein G | Alexa 546 | HEK or HCT 116 or A 431 | Yes |
| antibody | | | | cells, or human patient | |
| | | | | tumour | |
| Anti-TrkB | Alexa 488 | Protein G | Alexa 594 | HEK or HCT 116 or A 431 | Yes |
| antibody | | | | cells, or human patient | |
| | | | | tumour | |
| Anti-TrkB | Alexa 488 | Protein A | Alexa 546 | HEK or HCT 116 or A 431 | Yes |
| antibody | | | | cells | |
| Anti-TrkB | Alexa 488 | Protein A | Alexa 594 | HEK or HCT 116 or A 431 | Yes |
| antibody | | | | cells | |
| Anti-TrkB | Alexa 488 | Protein G | Alexa 546 | HEK or HCT 116 or A 431 | Yes |
| antibody | | | | cells, or human patient | |
| | | | | tumour | |
| Anti-TrkB | Alexa 488 | Protein G | Alexa 594 | HEK or HCT 116 or A 431 | Yes |
| antibody | | | | cells, or human patient | |
| | | | | tumour | |
| Anti-EGFR | Alexa 488 | Protein G | Alexa 546 | HEK or HCT 116 or A 431 | Yes |
| antibody (AY3 | | | | cells, or human patient | |
| clone) | | | | tumour | |
| Anti-EGFR | Alexa 488 | Protein G | Alexa 546 | HEK or HCT 116 or A 431 | Yes |
| antibody (MA- | | | | cells, or human patient | |
| 12693 clone) | | | | tumour | |
| Anti-EGFR | CF Dye 503R | Protein G | CF Dye 555 | HEK or HCT 116 or A 431 | Yes |
| antibody (MA- | | | | cells, or human patient | |
| 12693 clone) | | | | tumour | |
| Anti-EGFR | CF Dye 503R | Protein G | CF Dye 594 | HEK or HCT 116 or A 431 | Yes |
| antibody (MA- | | | | cells, or human patient | |
| 12693 clone) | | | | tumour | |
| Anti-EGFR | CF Dye 503R | Protein G | CF Dye 555 | HEK or HCT 116 or A 431 | Yes |
| antibody (MA- | | | | cells, or human patient | |
| 12693 clone) | | | | tumour | |
| Anti-EGFR | CF Dye 503R | Protein G | CF Dye 594 | HEK or HCT 116 or A 431 | Yes |
| antibody (MA- | | | | cells, or human patient | |
| 12693 clone) | | | | tumour | |

### Example 4: Fluorescent Antibody-Polypeptide Probe

### Fluorescent Probe Preparation

An organosilica (thiol silica)/zirconia sol is prepared 24 hours before depositing on the substrate: 20 mL of mercaptotriethoxysilane/zirconium chloride/ethanol/water mixture (0.95/0.05/40/5) is stirred at ambient temperature. The sol is deposited on a substrate, typically a 50-micron thick PET film, by dip-coating (at a withdrawal rate of 5.5 mm·s⁻¹), and is heat-treated at 80° C. for 12 hours. A first binding molecule, 1-(2-amino-ethyl)-pyrrole-2,5-dione hydrochloride, containing a maleimide group and an amine group is dissolved in DMSO at 0.1 mol·L⁻¹ then this solution is added onto the polymer film for 1 hour under stirring at ambient temperature. After rinses and washes with DMSO and with distilled water, a second solution containing another binding molecule, N-succinimidyl 4-maleimidobutyrate, containing an NHS ester group and a maleimide in DMSO (at 0.1 moL·L⁻¹) is added onto the polymer film for lh under stirring. After rinses and washes with DMOS and with distilled water, the substrate is ready to bind a polypeptide.

A solution of anti-EGFR antibody (labelled with Alexa488) is incubated with a polypeptide (labelled with Alexa546) of amino acid sequence as described in SEQ ID NO: 2 (CCGGRRGW), immobilised on the substrate for 60 minutes at ambient temperature.

A fluorescent probe comprising the same antibody bonded to a protein G is produced according to the same process. In this case, the protein G and the antibody carry the same fluorochromes as in the preceding case.

### Target Molecule Detection

After incubation, the spectral properties are measured with a fluorimeter, with an excitation at 488 nm.

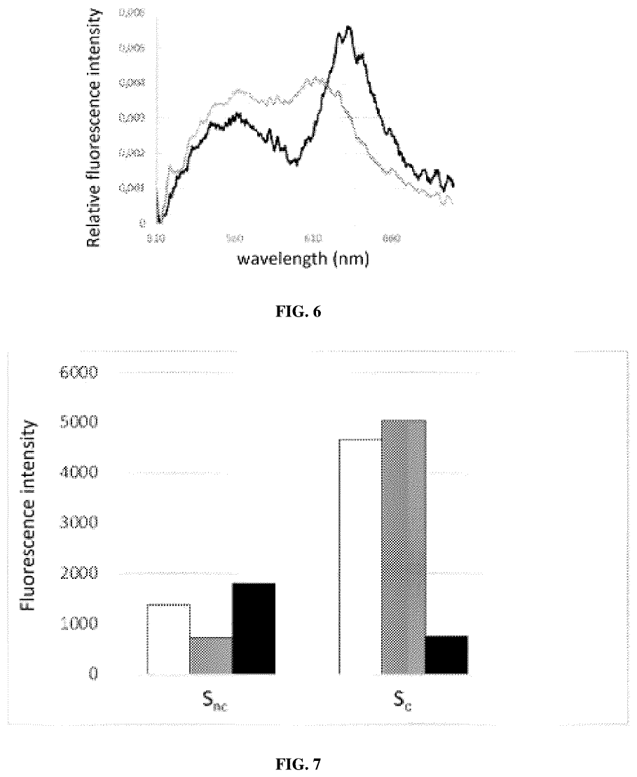

The two probes have different optical properties. Indeed, the FRET effect is greater between the antibody and the peptide than between the antibody and protein G, as illustrated in FIG. 6. This difference in fluorescence intensity can be explained by the different distance between the fluorochromes Fₐ and F_{b} in the two types of probes. The peptide chain of the peptide being shorter than that of protein G, the distance between the fluorochromes is also shorter.

### Example C1: Comparative Example 1—Covalent Antibody-Protein G Bond

### Preparation of a Fluorescent Probe Comprising an Antibody Bonded to a Protein G Via a Non-Covalent Bond—S_{nc}

A protein G (labelled with Alexa546) is grafted covalently at the bottom of a previously functionalised well plate, as described above. A solution of anti-EGFR antibody (labelled with Alexa488) at 10 μg/ml is incubated, in order to enable the adhesion of the antibody on protein G non-covalently. The excess antibody is washed with PBS.

### Preparation of a Fluorescent Probe Comprising an Antibody Bonded to a Protein G Via a Covalent Bond—S_{c}

A protein G (labelled with Alexa546) is grafted covalently at the bottom of a previously functionalised well plate, as described above. A 2 mM glutaraldehyde solution is added for 15 minutes. The excess glutaraldehyde is washed with PBS. A solution of anti-EGFR antibody (labelled with Alexa488) at 10 μg/ml is incubated, in order to enable the adhesion of the antibody on protein G covalently. The excess antibody is washed with PBS.

The fluorescence intensity emitted by the antibody of the two fluorescent probes S_{nc} and S_{c} was measured, after excitation at 488 nm.

### Evaluation of the Stability of the Antibody-Protein G Bond

Each fluorescent probe S_{nc} and S_{c} was then incubated in 100 μL of an aqueous 0.1M Glycine solution (pH 2.5) for 30 minutes at 37° C., which lowers the affinity of protein G for the antibody. After incubation, the glycine solution was deposited in an adjacent well.

The intensity of fluorescence of the antibody remaining assembled as well as that of the eluted antibody was measured. This makes it possible to determine the quantity of antibody which has detached from the binding protein, i.e. evaluate the strength of the antibody-protein bond and therefore the probability of separation of the fluorescent probe.

Adding the step of binding with glutaraldehyde increases the quantity of antibody associated with protein G, and induces a covalent bond between the two. FIG. 7 shows the relationship between the quantity of antibody successfully bound on protein G (white column), the quantity of antibody remaining attached to protein G after incubation with glycine (grey column) and the quantity of antibody eluted after incubation with glycine (black column), with or without a covalent bond. It is apparent that:
the quantity of antibody successfully bound with protein G is increased when a covalent bond is formed, i.e. the production yield of fluorescent probe that can be used is increased;
the quantity of antibody remaining attached to protein G is greater in the case of the covalent bond, attesting to the strength of this bond, which means that it is more likely that the fluorescent probe separates in the case of non-covalent bond, rendering this type of probe unusable in vivo;
after elution, the antibody is in solution. The fluorescent intensity detected in the eluent appears to be greater than the initial quantity for two reasons: it is no longer bonded to protein G which lowers the fluorescence intensity thereof via a FRET effect, and the laser excitation is no longer performed by a single layer of grafted proteins, but on a column of solution.

The fluorescent probe is therefore stabilised by the presence of a covalent bond between the antibody and the protein. The same phenomenon is observable in the case of smaller polypeptides.

In the case of the probe S_{c}, the quantity of “remaining” antibody is higher than the quantity of “initial” antibody, which is due to the margins of error of the measuring apparatus and in no way modifies the above conclusions.

### Target Molecule Detection

The FRET index (acceptor fluorescence intensity/donor fluorescence intensity) was measured using a fluorimeter after excitation at 488 nm for each fluorescent probe S_{nc} and S_{c}

The EGFR antigen diluted in PBS was added at a final concentration of 50 nM into each well containing the fluorescent probe solutions S_{nc} and S_{c}.

The FRET index (acceptor fluorescence intensity/donor fluorescence intensity) was measured after excitation at 488 nm for each fluorescent probe S_{nc} and S_{c} after adding the antigen and after one hour of incubation at ambient temperature. The amplitude of the change in response to this addition (“Fold change”) was measured with the FRET index with EGFR/FRET index without EGFR formula.

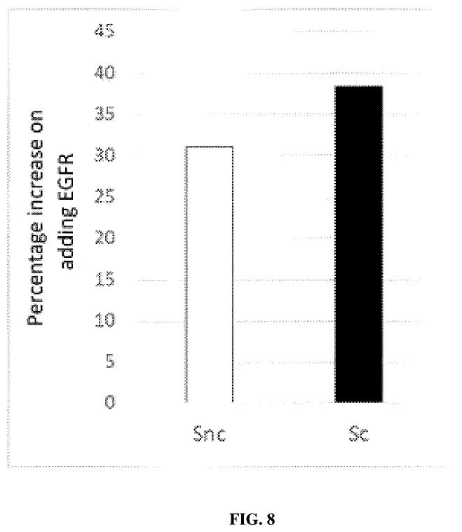

FIG. 8 shows the amplitude of this change in the case of the fluorescent probe S_{nc} (no covalent bond between antibody and protein) and the fluorescent probe S_{c} (covalent bond between antibody and protein). It is apparent that the presence of a covalent antibody-protein bond increases the amplitude of variation of the FRET index in response to adding antigen, increasing the sensitivity of the fluorescent probe.

### REFERENCE NUMBER

1—Fluorescent probe
11—Receptor
12—Polypeptide
2—Target molecule
3—Grafting molecule
4—Optical fibre
41—Port
42—Outer emission face
43—Body
44—Ferrule
45—Core
46—Cladding
5—Device
Fₐ—Fluorochrome bonded to the receptor (inactive configuration)
F′ₐ—Fluorochrome bonded to the receptor (active configuration)
F_{b}—Fluorochrome bonded to the polypeptide (inactive configuration)
F′_{b}—Fluorochrome bonded to the polypeptide (active configuration)
XX′—Longitudinal axis of the optical fibre

1-15. (canceled)

16. A device for detecting a target molecule and/or measuring the concentration of a target molecule comprising:
a substrate at the surface of which a grafting molecule is covalently attached;
at least one fluorescent probe comprising:
at least one receptor bonded to a polypeptide via a covalent bond;
two fluorochromes Fₐ and F_{b};
wherein the fluorochrome Fₐ is bonded to the receptor and the fluorochrome F_{b} is bonded to the polypeptide; and
the fluorochromes Fₐ and F_{b} form a FRET donor/acceptor pair;
wherein the polypeptide is bonded to the grafting molecule via a covalent bond.

17. The device according to claim 16, wherein the receptor is chosen from antibody, antibody fragment, aptamer, peptides, or a derivative thereof.

18. The device according to claim 16, wherein the polypeptide is a binding protein chosen from protein G, protein L, protein A, protein Z, protein M, immunoglobulin, a complete or partial immunoglobulin, or a derivative thereof.

19. The device according to claim 16, wherein the polypeptide comprises between 2 and 100 amino acids.

20. The device according to claim 16, wherein the fluorochromes Fₐ and/or F_{b} are chosen from fluorescent molecules or fluorescent proteins.

21. The device according to claim 16, wherein the substrate is chosen from a cell culture plate, a well plate, a film, a strip, an agarose gel, a cellulose gel, nanoparticles or microparticles, a microscope slide, a glass strip, the periphery of an optical fibre or a substrate configured to be attached to head of an optical fibre.

22. The device according to claim 21, wherein the substrate is a polymer film.

23. The device according to claim 22, wherein the polymer film is chosen from polyethylene terephthalate, fluorinated polyethylene-co-propylene, polymethylmethacrylate, polytetrafluoroethylene, polymethylpentene, polyvinyl chloride, styrene methyl methacrylate, polyethylene naphthalate, derivatives thereof or a mixture thereof.

24. The device according to claim 16, wherein the grafting molecule comprises at least two reactive groups chosen from maleimide, N-Hydroxysuccinimide (NHS) ester, sulfo N-hydroxysuccinimide ester, sulfo-NHS, azide, alkyne, epoxide, carboxylic acid, aldehyde, aziridine, alkene, or a derivative thereof.

25. The device according to claim 16, further comprising an optical fibre and an exploration head, wherein said exploration head comprises a body and an emission face, at least a part of which is transparent forming a port, the substrate being said port.

26. A fluorescent probe comprising:
at least one receptor bonded to a polypeptide via a covalent bond;
two fluorochromes Fₐ and F_{b};
wherein the fluorochrome Fₐ is bonded to the receptor and the fluorochrome F_{b} is bonded to the polypeptide; and
the fluorochromes Fₐ and F_{b} form a FRET donor/acceptor pair.

27. The fluorescent probe according to claim 26, wherein the receptor is chosen from antibody, antibody fragment, aptamers, peptides, or a derivative thereof.

28. The fluorescent probe according to claim 26, wherein the polypeptide is a binding protein chosen from protein G, protein L, protein A, protein Z, protein M, immunoglobulin, a complete or partial immunoglobulin, or a derivative thereof.

29. The fluorescent probe according to claim 26, wherein the polypeptide comprises between 2 and 100 amino acids.

30. A method for detecting a target molecule and/or measuring the concentration of a target molecule comprising the following steps:
placing a sample and at least one fluorescent probe in contact, said fluorescent probe comprising:
at least one receptor bonded to a polypeptide via a covalent bond;
two fluorochromes Fₐ and F_{b};
wherein the fluorochrome Fₐ is bonded to the receptor and the fluorochrome F_{b} is bonded to the polypeptide; and
the fluorochromes Fₐ and F_{b} form a FRET donor/acceptor pair; and
the receptor has an affinity for said target molecule;
exciting the fluorescent probe at a given wavelength such that the donor fluorochrome is excited;
measuring the ratio between the intensity of the fluorescence emitted by the donor fluorochrome and the intensity of the fluorescence emitted by the acceptor fluorochrome; and
determining the presence or the absence of said target molecule in the sample and/or calculating the concentration of said target molecule in the sample.

30. The device according to claim 16, wherein the polypeptide comprises between 4 and 50 amino acids.

31. The fluorescent probe according to claim 26, wherein the polypeptide comprises between 4 and 50 amino acids.
